(19) United States
(12) Patent Application Publication
(10) Pub. No.: US 2007/0059198 A1
(43) Pub. Date: Mar. 15, 2007

(54) Anti-bacterial syringe and associated reservoir

(76) Inventors: Stephan Donald Prestwood, Alpharetta, GA (US); Marc Harlan Minter, Duluth, GA (US); Jay Alexander Donohue, Flowery Branch, GA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/258,789

(22) Filed: Oct. 26, 2005

(30) Foreign Application Priority Data
Jul. 29, 2005 (EP) 2005/008272

(51) Int. Cl. A61L 2/00

(52) U.S. Cl. 422/24

(57) ABSTRACT

This invention is related to an apparatus and method for filling a smaller container with a fluid from a reservoir that minimizes microbial growth.

### BACKGROUND

Many manufacturing processes require an automated process for automatically dispensing a fluid in a prescribed amount. These fill processes typically involve filling a smaller container with liquid from a larger container or reservoir. Part of this process entails sensing the amount of fluid present in the reservoir to determine when the reservoir needs to be refilled. Another component is providing a connection from the reservoir to the dispenser to allow fluid flow.

Some industries, such as, for example, the medical device or pharmaceutical industry, are regulated by the FDA for safety and efficacy. As such, the manufacturing process used in these industries must produce a product that can be safely delivered to a consumer in a sterile form. For these types of processes, any medium, such as a liquid or gas, may be used to for packaging, hydration or extraction. In some situations, the medium may not be sterile, and as such may be a source of contamination. In these situations, it is particularly important to have a precisely controlled fill process that controls the potential for microbial growth. This fill process may be a part of the final packaging process or may be an intermediary component of the manufacturing process, such, as for example, part of the transport cycle or extraction process.

Ophthalmic lenses are considered medical devices and as, such, require very precise manufacturing processes. Such processes are described, for example in U.S. Pat. No. 6,113,817, which is expressly incorporated by reference as if fully set forth herein. Ophthalmic lenses may be made in many ways, the majority of which are molding processes. In a typical molding system, lenses are cycled through a series of stations on a semi-continuous basis. The cyclic portion of lens production generally involves dispensing a liquid crosslinkable and/or polymerizable material into a female mold half, mating a male mold half to the female mold half, irradiating to crosslink and/or polymerize, separating the mold halves and removing the lens, packaging the lens, cleaning the mold halves and returning the mold halves to the dispensing position.

Soft hydrophilic contact lenses are generally manufactured from hydrophilic polymer material. Depending on the composition of the polymer, the lenses may have a water content of from 20 percent to 90 percent or more. Such contact lenses must be preserved and stored in a liquid such as a sterile aqueous solution, usually an isotonic saline solution, to prevent them from drying out and to maintain them in a state ready for use.

The packaging process entails filling a small container with a predetermined amount of liquid from a reservoir, adding a lens to the smaller container, and sealing the smaller container. Generally the smaller container is filled with a non-sterile solution. During this process, contamination of the reservoir, the connection between the reservoir, the mobile dispenser, or the smaller container is possible. This problem is exacerbated by the fact that the components may be difficult to clean or disinfect. The present invention seeks to prevent such contamination while improving the precision of the fill process.

### SUMMARY OF THE INVENTION

The present invention seeks to solve the problems listed herein by providing an apparatus for dispensing a fluid that comprises a syringe bracket, one or more syringes attached to the bracket, a reservoir in fluid connection with one or more syringes and a UV light source that is in optical connection with the reservoir. The UV light source is preferably used to irradiate the fluid, thereby minimizing microbial growth. In one embodiment, the apparatus may include five syringes. In another embodiment a syringe may comprise a syringe barrel, a syringe tip, a plunger rod, and a plunger rod flange.

In one embodiment of the present invention, the syringes may be disposable. In another embodiment, the reservoir may be adapted to allow overflow of the fluid. In a preferred embodiment, the reservoir includes a non-opaque bottom panel. Onther embodiments may include sensors that are adapted to sense the presence of air bubbles in the syringes and/or the fluid level in the reservoir.

In one embodiment, the reservoir is made from stainless steel. In another embodiment, the reservoir is adapted to move between at least two positions. In a related embodiment, the movement of the reservoir is accomplished by actuators. In another embodiment, the syringe bracket is adapted to move between at least two positions. In a related embodiment, actuators may be used to move the syringe bracket.

In one embodiment, the reservoir has a cover. In a related embodiment, the cover inhibits the formation of salt crystals. In still another embodiment, the cover may have one or more holes for the syringes.

The present invention also includes a method for dispensing a fluid that entails filling a reservoir with fluid, moving a syringe bracket and associated syringes in close proximity to the reservoir, placing the syrignes over the reservoir, engaging the barrel flanges of the syringes, drawing fluid into the syringes, moving the syringe bracket and associated syringes over a manufacturing line, and dispensing the fluid. This method may also include exposing the fluid in the reservoir to UV light. In one embodiment the method may be repeated. In still another embodiment, the syringes may be replaced.

These and other aspects of the invention will become apparent from the following description of the preferred embodiments taken in conjunction with the following drawings. As would be obvious to one skilled in the art, many variations and modifications of the invention may be affected without departing from the spirit and scope of the novel concepts of the disclosure.

### BRIEF DESCRIPTION OF THE DRAWINGS

FIG. 1 illustrates a plan view of one embodiment showing all three components of the present invention.

FIG. 2 depicts a syringe used in one embodiment of the present invention.

FIG. 3 depicts a syringe bracket used in one embodiment of the present invention.

FIG. 4 depicts a reservoir of the present invention.

FIG. 5 depicts a front view of a syringe bracket used in one embodiment of the present invention.

FIG. 6 depicts a side view of the syringe bracket shown in FIG. 5.

### DETAILED DESCRIPTION OF THE PREFERRED EMBODIMENTS

Reference now will be made in detail to the embodiments of the invention. It will be apparent to those skilled in the art that various modifications and variations can be made in the present invention without departing from the scope or spirit of the invention. For instance, features illustrated or described as part of one embodiment, can be used on another embodiment to yield a still further embodiment. Thus, it is intended that the present invention cover such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention. All patents and patent applications disclosed herein are expressly incorporated by reference in their entirety.

Unless defined otherwise, all technical and scientific terms used herein have the same meaning as commonly understood by one of ordinary skill in the art to which this invention belongs. Generally, the nomenclature used herein and the laboratory procedures are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well known and commonly employed in the art.

An “ophthalmic device,” as used herein, refers to a contact lens (hard or soft), a corneal onlay, implantable ophthalmic devices used in, on or about the eye or ocular vicinity. The term “contact lens” employed herein in a broad sense and is intended to encompass any hard or soft lens used on the eye or ocular vicinity for vision correction, diagnosis, sample collection, drug delivery, wound healing, cosmetic appearance (e.g., eye color modification), or other ophthalmic applications.

The term “fluid” as used herein indicates that a material is capable of flowing like a liquid.

The present invention provides to an apparatus and method for filling a smaller container with a fluid from a reservoir that minimizes microbial growth. The apparatus and method involve use of a “filler” syringe, preferably a disposable syringe. The reservoir is preferably adapted to allow an optical connection between the fluid in the reservoir and UV light, which may also act to prevent microbial growth. The fluid in the reservoir may be any type of fluid that supports the growth of microorganisms. For example, in a specific embodiment, the fluid may be saline, a preservative, or a buffer for removing excess material from ionic contact lenses. Use of UV light to kill microorganisms or inhibit microorganism growth is known in the art.

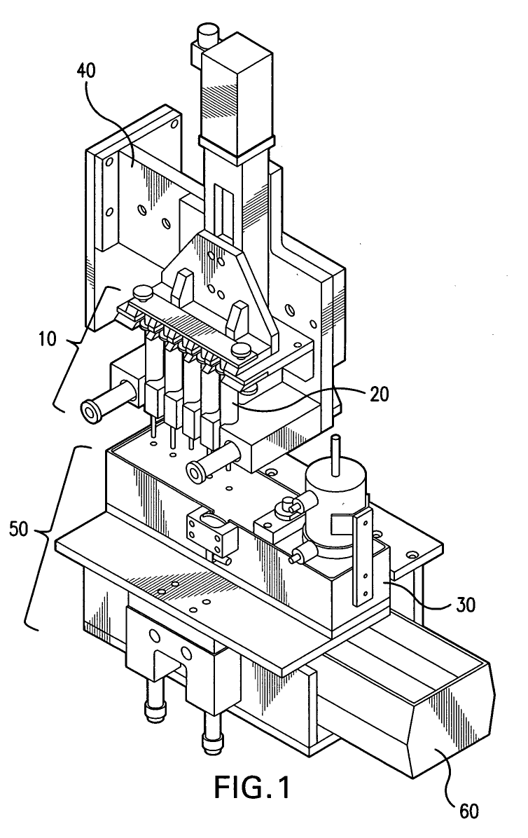

Referring to FIG. 1, the syringe filler is made of three basic components, a syringe bracket 10, one or more syringes 20, and a reservoir 30. Syringe bracket 10 is preferably connected to a movable fixture 40 that may be attached to any component on a manufacturing line. One or more syringes 20 preferably hang from syringe bracket 10 and are in fluid connection with reservoir 30. In a preferred embodiment, there may be a plurality of syringes adapted to be placed directly over a plurality of smaller containers. In a more specific embodiment, the smaller containers may be individual contact lens packages. Reservoir 30 is preferably attached to reservoir fixture 50. Reservoir fixture 50 preferably affixes a UV light source 60 in optical connection with reservoir 30. Reservoir 50 may be of any orientation or support structure that is adapted to support reservoir 30 and UV light source 60. Reservoir fixture 50 is preferably capable of movement in vertical and/or horizontal direction. Such movement may be controlled or facilitated by one or more slides or actuators.

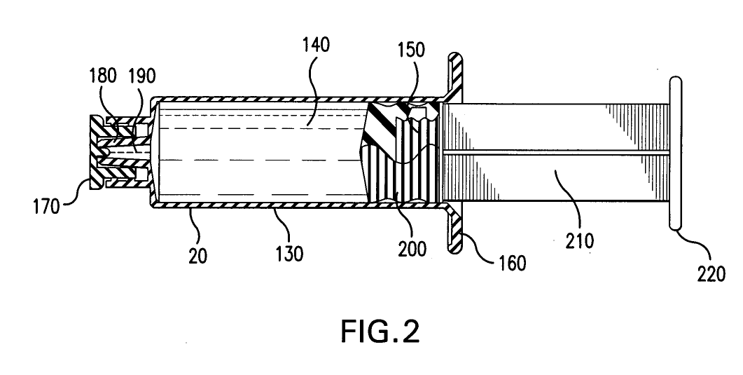

A typical syringe which may be used in conjunction with the present invention is shown in FIG. 2. A syringe 20 of the present invention preferably includes a syringe barrel 120 having an elongated body 130 defining a chamber 140 for retaining fluid. The syringe barrel may include an open proximal end 150 having a barrel flange 160, a distal end 170 and a frusto-conical shaped end 180 extending from the distal end and having a tip passageway 190 in fluid communication with the chamber. The chamber may have a generally constant diameter of constant cross-section portion of the inside of the barrel. A stopper 200 is in fluid-tight engagement inside the barrel. An elongated plunger rod 210 may extend proximally from the stopper through the open proximal end of the barrel. In this embodiment, plunger rod 210 and stopper 200 are separate elements. Stopper 200 is preferably made of an elastomeric material selected from the group of natural rubber, synthetic rubber, thermoplastic elastomers or combinations thereof. In another preferred embodiment, an elastomeric stopper may not be present in the interior of the syringe, but rather may have an interference fit between the inside of the syringe barrel and a sealing lip on the end of the plunger. The plunger rod may also include a plunger rod flange 220 at the proximal end of the plunger rod.

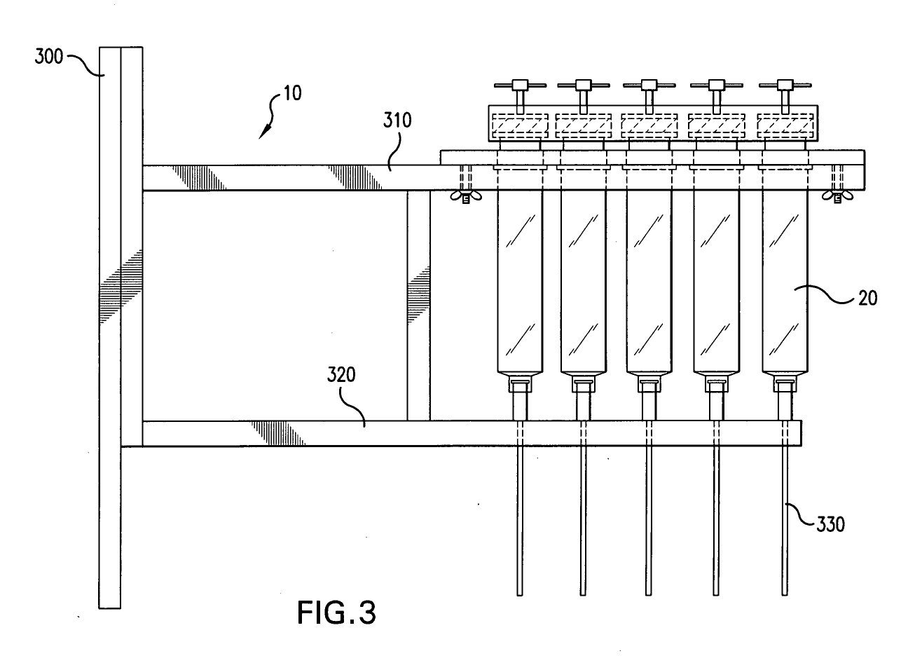

In a more preferred embodiment the syringe bracket 10 is adapted to receive a Henke Sass Wolf GmbH (HSW) (Tuttlingen, Germany) Norm-Ject AL20 syringe. The present invention preferably includes a syringe 20 that has an applicator tip 330 as shown in FIG. 3. In a preferred embodiment tip 330 is a stainless steel applicator tip. For example, an acceptable tip may be a JG12-2.0 tip from Jensen Global (Santa Barbara, Calif.). The syringes used in conjunction with the present invention are preferably disposable, which prevents excess growth of microorganisms and other undesirables. After a specified number of fills, each syringe may be removed and replaced with a new, sterile syringe. In one embodiment, the syringes may be replaced about every 24 hours. In another embodiment the syringes may be replaced about once a week.

Referring to FIG. 3, the syringe bracket 10 is preferably mounted to an existing component 300 that may be an existing transverse slide or actuator. Component 300 is preferably capable of movement in vertical and/or horizontal direction. The existing component 300 is preferably adapted to allow the syringe bracket 10 and syringes 20 to index directly over a small container. In another preferred embodiment, the syringes 20 are adapted to move with components on a manufacturing line or similar automated process. The syringe bracket preferably consists of at least two plates (upper plate 310 and lower plate 320) that extend horizontally from existing component 300. Upper plate 310 is preferably designed to contact barrel flange 160 of syringe 20 by preventing slippage of the syringe past upper plate 310.

Syringe 20 preferably extends through an aperture in upper plate 310. The aperture (not shown) preferably has a diameter large enough to accommodate syringe barrel 120, while having a diameter small enough to prevent barrel flange 160 from sliding through the aperture. Lower plate 320 is preferably designed to contact frusto-conical shaped end 180 of syringe 20 by preventing slippage of the syringe past lower plate 320. Tip 330 preferably extends through an aperture in lower plate 320. The aperture in lower plate 320 (not shown) preferably has a diameter large enough to accommodate tip 330, while having a diameter small enough to prevent frusto-conical shaped end 180 of syringe 20 from sliding through the aperture.

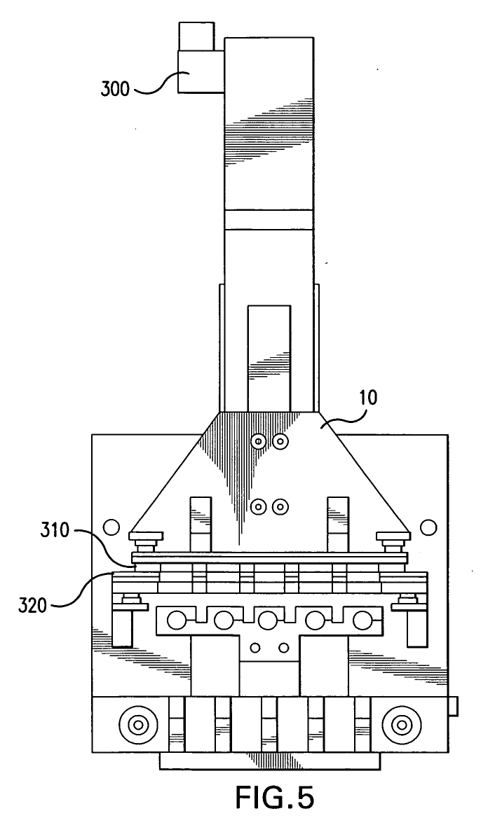

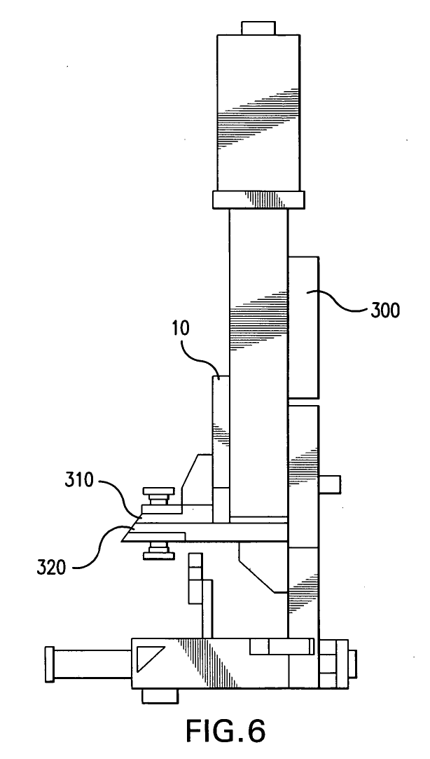

FIGS. 5 and 6 depict an alternative embodiment of the syringe bracket utilized in the present invention. As previously discussed, the syringe bracket 10 is preferably mounted to an existing component 300 that may be an existing transverse slide or actuator. Component 300 is preferably capable of movement in vertical and/or horizontal directions. The existing component 300 is preferably adapted to allow the syringe bracket 10 and syringes 20 to index directly over a small container. FIGS. 5 and 6 both use at least two plates, 310 and 320, to draw fluid into and release fluid from the syringes.

In another embodiment, the syringes may be gripped by one or more clamps. These clamps preferably grip the barrel flange 160. A second series of clamps may also be used to grip the flange and the end of the syringe plunger, which may be mounted to a vertical slide or actuator, such as for example, a Berger Lahr vertical slide. In a preferred embodiment, a set of grasp plates in conjunction with upper plate 310 are used to grasp and control the syringes. A first grasp plate 340 may be affixed horizontally over the tip of the syringe plunger. A second grasp plate 350 is preferably affixed underneath the syringe flanges. The first and second grasp plates 340 and 350, when actuated, are adapted to move vertically, thus moving the plunger flange up or down to dispense fluid. All plates and other components of the invention may be chamfered or have rounded edges to remove any sharp edges or corners.

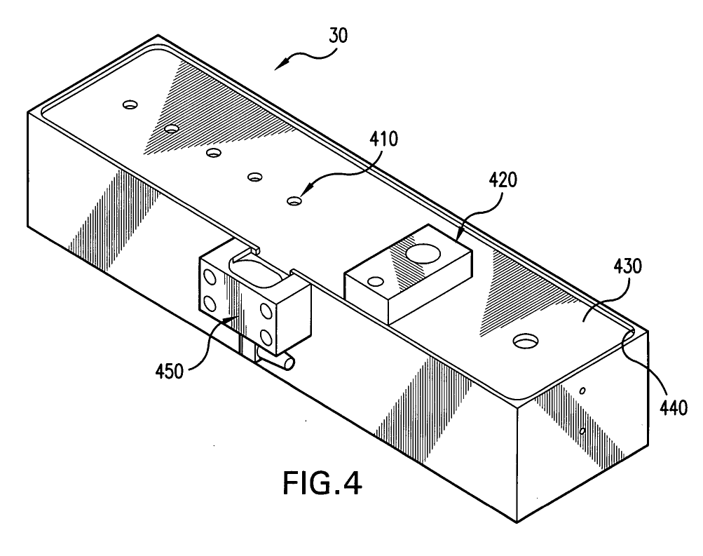

The third component of the syringe filler is reservoir 30, which is shown in detail by FIG. 4. Reservoir 30, which preferably resides on the side of the manufacturing line, preferably in a stable position on reservoir fixture 50, is shown in FIG. 1. Reservoir 30 preferably consists of a rectangular-shaped reservoir with a bottom panel that allows transmission of light. Other shapes may be used in alternative embodiments. In a preferred embodiment, the reservoir is made of stainless steel, such as, for example, 316L stainless steel. In another preferred embodiment, the bottom panel is made of a non-opaque material, such as for example, glass, more preferably quartz glass capable of allowing 254 nm UV light to pass through it. Reservoir cover 430 is preferably adapted to allow fluid to flow from the reservoir into the syringes via a plurality of fill holes 410 in cover 430. The number of fill holes 410 preferably corresponds to the number of syringes used. Additionally, cover 430 reservoir may be adapted for a sensor, preferably a capacitance-type level sensor. For example, as shown in FIG. 4, an elevated block 420 may be bolted to reservoir 30 to hold a sensor. In a preferred embodiment, the sensor signals a pump, such as a peristaltic pump, for example, to fill the reservoir. The top of the reservoir may also be adapted to have an orifice for tubing that provides fluid communication between reservoir 30 and a fluid source, which allows refill of the reservoir. As stated previously, this connection may include an air gap to prevent cross-contamination between the fluid source and the reservoir. A low pressure UV fixture may also be mounted to the bracket that holds the reservoir as shown in FIG. 1.

Cover 430 is designed to prevent contaminants from entering reservoir 30 and may also be adapted to prevent overflow. Overflow may be prevented by a recess 440 in cover 420. In preferred embodiment recess 440 is preferably about ⅛ of an inch. Additionally, an overflow catch basin 450 may be attached to reservoir 30 to catch any overflow. Cover 430 also aids in preventing formation of salt crystals or other undesirables in waste or overflow channels.

Reservoir 30 may be adapted to move between two distinct positions via reservoir fixture 50 and its associated actuators and/or slides. The initial reservoir position is preferably a retracted position in which reservoir 30 is filled with a fluid. The second position is preferably a position in which the reservoir moves toward the manufacturing line and up towards the syringe bracket to allow fill of the syringes. The syringes and associated bracket preferably have a plurality of positions in a horizontal plane that may also be controlled and facilitated by an actuator. The home position for the syringe is preferably directly over the reservoir. From that position, the syringes and associated brackets are adapted to move over the manufacturing line. The syringes and associated syringe bracket are adapted to move to various positions about the manufacturing line to fill smaller containers. In one embodiment, the syringes and syringe bracket move to a point at a maximum distance from home position and index over smaller containers back to the home position, dispensing a specified amount of fluid into each smaller container.

The system of the present invention is designed to maintain a substantially uniform continuous flow of liquid from reservoir 30 into one or more smaller containers. In a preferred embodiment, this system prevents microbial growth. The system preferably begins when the reservoir is empty. When a level sensor in the reservoir indicates that there is insufficient fluid in the reservoir, a pump, such as for example, a peristaltic pump, may draw fluid from another container into the reservoir by means of an orifice provided for this purpose in the top of the reservoir. In a specific embodiment applicable to contact lens manufacture, the pump may draw saline from a saline bag. During this operation, the reservoir is preferably in a retracted position, away from the syringes and syringe brackets. After the reservoir is filled, the syringe bracket is then free to travel to the home position above the reservoir. The reservoir may then extend to meet the syringe bracket, allowing the applicator tips on the syringe to enter the plurality of orifices in the top of the reservoir. The applicator tips preferably extend below the level of fluid in the reservoir. The vertical slide and/or actuator, then lifts the syringe plungers to the topmost position, which is fully extended, thus drawing fluid from the reservoir into the syringes. The reservoir can then retract, providing clearance for the syringe bracket to index over the first strip of five primary packages. Once in position, the vertical slide/ actuator depresses the plungers on all five syringes, dispensing a predetermined quantity of fluid into the smaller containers. In one embodiment of the present invention the smaller containers are contact lens wells, which are part of the contact lens packaging. This repeats, with the syringe bracket indexing over the next strip of smaller containers, dispensing fluid into them until the syringes are empty. The syringe bracket then returns to the home position where the saline reservoir rises to meet the syringes once again to refill them.

The present invention may also be equipped with one or more sensors to facilitate automation. In one embodiment, a sensor is used in conjunction with the reservoir to determine the fluid level of the reservoir. In another embodiment, one or more sensors may be used in conjunction with one or more syringes to determine whether air bubbles are present in the fluid inside the syringes. In each embodiment using a sensor, an undesirable condition, such as a bubble in a syringe, for example, may generate an error message to the operator. In another embodiment, an undesirable condition may stop operation of the manufacturing process or machine. A preferred sensor is a capacitance sensor, but any sensor may be used. Additionally, each actuator used in the present invention may have one or more sensors associated with it. In these embodiments, each sensor is used to determine whether that actuator has reached its vertical or horizontal limit.

In a preferred embodiment, the components of the present invention are made from anodized aluminum or an equivalent material. The needles, lower plate 320, reservoir 30, and reservoir cover 430 are preferably made from stainless steel or any equivalent material that prevents corrosion. Any elastomers used are preferably ethylene propylene (EDPM) or another similar material that can withstand UV light.

What is claimed is:

1. An apparatus for dispensing a fluid comprising
a syringe bracket,
one or more syringes fixedly attached to said syringe bracket;
a reservoir adapted to be in fluid connection with said one or more syringes, and
a UV light source, wherein said light source is in optical connection with said reservoir to irradiate the fluid, thereby minimizing microbial growth.

2. The apparatus of claim 1, further comprising five syringes.

3. The apparatus of claim 1 wherein said one or more syringes further comprise
a syringe barrel;
a syringe tip;
a plunger rod; and
a plunger rod flange.

4. The apparatus of claim 1, wherein said one or more syringes are disposable.

5. The apparatus of claim 1, wherein said reservoir is adapted to allow overflow of fluid.

6. The apparatus of claim 1, wherein said reservoir further comprises a non-opaque bottom panel.

7. The apparatus of claim 1, further comprising a plurality of sensors.

8. The apparatus of claim 7, wherein one of said plurality of sensors senses the fluid level in said reservoir

9. The apparatus of claim 7, wherein one of said plurality of sensors senses the presence of air bubbles in said syringes.

10. The apparatus of claim 1, wherein said reservoir is made of stainless steel.

11. The apparatus of claim 1, wherein said reservoir is adapted to move between at least two positions.

12. The apparatus of claim 11, wherein said movement is provided by one or more actuators.

13. The apparatus of claim 1, wherein said syringe bracket is adapted to move between at least two positions.

14. The apparatus of claim 13, wherein said movement is provided by one or more actuators.

15. The apparatus of claim 1, wherein said reservoir further comprises a cover.

16. The apparatus of claim 15, wherein said cover inhibits formation of salt crystals.

17. The apparatus of claim 15, wherein said cover is adapted to have one or more holes to accommodate a syringe.

18. The apparatus of claim 15, wherein said cover prevents evaporation.

19. A method for dispensing a fluid comprising:
filling a reservoir with fluid;
moving a syringe bracket and associated syringes in close proximity to said reservoir;
placing said syringes over said reservoir;
engaging the barrel flanges of said syringes;
drawing fluid into said syringes;
moving said syringe bracket and associated syringes over a manufacturing line; and dispensing said fluid.

20. The method of claim 19, further comprising exposing said fluid in said reservoir to UV light.

21. The method of claim 19, wherein said method is repeated.

22. The method of claim 18, wherein said syringes are disposable

23. The method of claim 19, further comprising replacing said syringes after a prescribed period of time.
